(19) United States
(12) Patent Application Publication
(10) Pub. No.: US 2025/0146026 A1
(43) Pub. Date: May 8, 2025

(54) CONGENER REMOVAL IN ETHANOL PRODUCTION

(71) Applicant: Whitefox Technologies Limited, London (GB)

(72) Inventors: Virginia Andrade, Calgary (CA); Stephan Rudiger BLUM, Calgary (CA); Gilmar ARENDS, Calgary (CA); Jessica LOPEZ, Calgary (CA)

(73) Assignee: Whitefox Technologies Limited, London (GB)

(21) Appl. No.: 18/938,780

(22) Filed: Nov. 6, 2024

Related U.S. Application Data
(60) Provisional application No. 63/547,783, filed on Nov. 8, 2023.

(51) Int. Cl. C12P 7/06

(52) U.S. Cl. CPC C12P 7/06

(57) ABSTRACT

Congener removal in ethanol production may be provided by fermenting a feedstock in a fermentation vessel, yielding a fermentation product including an organic solvent and water, and inert fermentation gases; distilling the fermentation product in a distillation column yielding a first solvent enriched stream; dehydrating the first solvent enriched stream yielding a second solvent enriched stream having a greater concentration of the organic solvent than the first solvent enriched stream and a water enriched solution; and sparging the water enriched solution with the inert fermentation gases in a sparging tank yielding a sparged solution and a vapor including volatile species present in the water enriched solution separated out from the sparged solution.

### CROSS-REFERENCE TO RELATED APPLICATION

The present application claims the benefit of and priority to U.S. Provisional Patent Application No. 63/547,783 titled “CONGENER REMOVAL IN ETHANOL PRODUCTION” and filed on Nov. 8, 2023, which is incorporated herein in its entirety.

### BACKGROUND

In the production of ethanol, typical processes use a series of distillation steps combined with dehydration units to be able to effectively separate water from ethanol produced from fermentation.

### SUMMARY

The present disclosure provides a bubble evaporation process and system for congener removal in ethanol production, given by a process configuration to remove low boiling and high boiling organic compounds from fuel grade or high-grade ethanol streams using inert gasses (e.g., carbon dioxide (CO₂)) that might already exist at the plant as the gas source through a system comprising a blower, a tank with sparging network, and a vent recovery system.

In the production of ethanol, typical processes use a series of distillation steps combined with dehydration units to be able to effectively separate water from ethanol produced from fermentation. The fermentation product (called beer or wine) consisting of primarily water with ethanol, solids, volatile compounds (e.g., acetaldehyde, ethyl acetate and odor species), fusel oils (e.g., C3, C4 and C5 alcohols), and other high boiling compounds is fed to a distillation column to concentrate the ethanol up to between 90 proof (90P) to 130 proof (130P) by removing water and solids as a bottom product. The ethanol first distillate (now containing more concentrated ethanol, fusel oils, and volatile species) is subsequently fed to a rectifier column. The rectifier overheads vapor (often 190 proof or 190P) from the stripper/rectifier unit consisting of primarily ethanol (˜90 vol-% ethanol) and high quantities of low boiling species is then fed to dehydration systems, such as membrane units and molecular sieve units (MSUs), for further dehydration producing >99 vol-% ethanol product (200 proof or 200P). In the MSU dehydration operation, water is removed by an adsorption mechanism with zeolite beads. This dehydration process requires two or more MSU beds as the beds will be cycling through a regeneration mode and a dehydration mode. During the regeneration mode, water is desorbed from the saturated zeolite pellets, creating a regenerate stream (Regen) comprising an ethanol concentration between 40 and 80 vol-% with large quantities of volatile components included in some situations. Higher concentration of volatile species in the regenerate stream might cause potential accumulation of the low boiling components by the recycle of regen to the distillation section. The accumulation of these species may lead to issues in the scrubber system and also may increase the difficulty to attain a final product that adheres to the concentration specifications desired. Additionally, in some cases, low boiling components can be generated in MSU bed via chemical reactions.

To recover the ethanol in the regenerate stream, a different separation system may be used comprising of a distillation column (e.g., a stripper column, a rectifier column) and a membrane system. This system forms a retentate stream (dehydrated product >99 vol-%) and a water-rich byproduct stream called the permeate (containing about 1 to 80 vol-% ethanol). The feed to the stripper column may consist of a combination of the regenerate and permeate streams, the regenerate, the permeate, 190P streams, 120P streams, rectifier bottoms, side stripper bottoms streams, distillation column draws, and low proof streams (e.g., of less than 120P, 110P, 100P, 90P, 80P, 70P, 60P, etc.), which can be fed to a feed tank before being fed to the stripper column; can be mixed in a feed tank before being fed to the stripper column; or can be fed directly to the stripper column. The volatile or low boiling compounds will appear in the stripper overheads feeding into the membrane unit.

In the production of ethanol, particularly for “high-grade” production for use in medicine, hand sanitizer, chemical production, and scientific purposes versus for use in fuel, the complete removal of the volatile, high boiling, and odor species deriving from the fermentation process is desired. One possibility for high-grade ethanol production is to include a low boilers removal operation for the water-rich regen before the stream is pushed towards the separation system. The inclusion of a low boilers removal operation prior to the dehydration operation is done in order to inhibit the side production of acetal from the dehydration of ethanol, which is difficult to remove as more of the compound is produced. In situations where the accumulation of low boiling species is still present, the energy input required to reduce the concentrations of the low boiling species by conventional distillation is increased substantially. One could design a much taller low boilers column, however, the costs involved make such a solution unfeasible. In situations where high concentrations of volatile odor species persist, one could introduce copper packing in the distillation columns to remove or diminish the formation of these compounds over time. However, results are unreliable and there are additional costs to install and maintain the packing material. Additionally, the packing material (e.g., copper packing) is consumed during removal operations, and therefore needs to be frequently replaced and is an operating cost (and maintenance issue) for plants that use such sacrificial packing materials.

One way to remove volatile species from the ethanol streams is to vent out these compounds from the tanks; however, given the affinity of some of these species to ethanol and/or water, one would need to i) design tanks with very large surface areas or ii) introduce a very high degree of agitation in these tanks to be able to reduce the concentration of these species making it unreasonable. In addition, very long residence times are required for this venting action to be possible, which make this approach impractical in many situations. Moreover, by venting the volatile species from the tanks, the solvent included in the tanks may also be vented, and the losses of the desired solvent may make production less efficient.

An alternative solution to remove low boilers from ethanol streams is to use several distillation columns and, in some cases, use further extractive distillation techniques, which makes the process both energy and capital intensive. The present disclosure therefore provides a more practical approach for addressing the accumulation of volatile species in the ethanol streams, which can be integrated into an already existing ethanol production facility without significantly affecting the overall process. The method includes generating bubbles through the sparging of an inert gas in a liquid feed tank (e.g., regen, permeate, 190P, 120P, distillation bottom, low proof streams, etc.) containing the volatile species as a pre-processing step. An integrated configuration of the method may significantly reduce costs and provide high levels of energy savings. The method provides the following advantages over conventional distillation processes: The bubbling mechanism produces high interfacial areas and effective mass transfer between the liquid and gas phase by continuously producing fresh gas-liquid interfaces. This effective mass transfer promotes and enhances the evaporation of the volatile species at temperatures lower than the boiling points thereof, which reduces the energy requirement of the low boilers column or even precludes the need to include a low boilers column in the processing for high-grade ethanol production.

Vigorous mixing through bubble generation also further enhances mass and heat transfer providing efficient removal of the volatile species at shorter residence times as equilibrium at the exiting saturated vapor is quickly reached.

The method is particularly useful for streams running at lower temperature ranges (˜30-60 degrees Celsius (C)) as the temperature will be well below the saturation temperature of ethanol, inhibiting high ethanol losses while still removing the low boiling species (e.g., acetaldehyde, hydrogen sulfide, carbon disulfide, methyl mercaptan, ethyl mercaptan, methanol, ethyl acetate, dimethyl sulfide, among other compounds known to those of skill in the relevant art.). As used herein, “low boiling species” or “low boilers” and related terms refer to compounds with a lower boiling point than a solvent product to be produced by a plant (e.g., ethanol). Similarly, as used herein, “high boiling species” or “high boilers” and related terms refer to compounds with a higher boiling point than a solvent product to be produced by a plant (e.g., ethanol).

The process can use a side product gas from ethanol fermentation as the main gas source. However, various gas mixtures (from various sources) may be used in addition to or instead of side product gasses. The gas mixture used for sparging is inert relative to the solvent produced by the plant, and may be composed of CO₂. In some embodiments, the gas mixture may come from fermentation, fermentation plus other vents from the plant before the gases are sent to the scrubber system (e.g., as pre-scrubber gases), vents after treated on the scrubber system (e.g., as post-scrubber gases), external source (outside of the plant), and the like.

In high-grade ethanol production processes, the method removes or reduces the need to supplement distillation columns or other vessels with copper packing for the reduction of odor components.

The method is also able to remove species with boiling points close to that of ethanol (e.g., within ±10 degrees C., more preferably within ±5 degrees C., more preferably within ±2.5 degrees C., or more preferably within ±1 degree C.).

The process is modular and can be easily adjusted to increase the removal of the volatiles by adjusting the sparging capacity, process temperature, and pressure conditions. One can even use different sources of gas for the sparging method.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates an example solvent production plant for an organic solvent, such as an alcohol, such as ethanol, according to aspects of the present disclosure.

FIGS. 2A-2B illustrate example components of a feed stripping section, as may be used in the solvent production plant described in FIG. 1, according to embodiments of the present disclosure.

FIGS. 3A-3C illustrate example components of a rectifying distillation section, as may be used in the solvent production plant described in FIG. 1, according to embodiments of the present disclosure.

FIGS. 4A-4G illustrate example components of a dehydration section, as may be used in the solvent production plant described in FIG. 1, according to embodiments of the present disclosure.

FIG. 5 illustrates example components of an evaporation section, as may be used in the solvent production plant described in FIG. 1, according to embodiments of the present disclosure.

FIGS. 6A-6G illustrate example embodiments of a dehydration section including a sparging tank, as may be used in the solvent production plant described in FIG. 1, according to embodiments of the present disclosure.

FIG. 7 illustrates a sparging tank, according to embodiments of the present disclosure.

FIGS. 8A-8D illustrate various embodiments of sparging manifolds, according to embodiments of the present disclosure.

FIG. 9 illustrates an inert gas sourcing scheme for use in congener removal in ethanol production, according to embedment's of the present disclosure.

FIG. 10 illustrates a flowchart of a method for congener removal in ethanol production, according to embodiments of the present disclosure.

For case of understanding, the elements shown in the Figures are presented in a schematic form familiar to those of ordinary skill in the art. Generally, this schematic form shows the elements in an orientation on the page that places the upper portion of those elements (as installed in operation) closer to the top of the page than elements corresponding to lower portions of those elements. The positioning of different elements (and the routing of pipes therebetween) within the plant relative to one another, however, will be understood to not necessarily conform to the elevation or arrangement shown on the page; elements may be grouped together or separated on the page for case of understanding or the effective use of space on the page without reference to the physical spacing of those elements in operation, unless stated otherwise.

### DETAILED DESCRIPTION

The present disclosure contemplates that one of ordinary skill in the art will be familiar with the various grades of the products of the system referred to herein. For the avoidance of doubt, ethanol is referred to by various proof levels of 100 proof (100P), 120 proof (120P), 190 proof (190P), and 200 proof (200P) that define purity levels for approximately at least 50% ethanol by volume, at least 60% ethanol by volume, approximately at least 95% ethanol by volume, and at least 99% ethanol by volume, respectively, but other purity levels may be specified for use according to the present disclosure. The present disclosure contemplates that different jurisdictions and technical field have standards that grade ethanol (and the additives or impurities therein) for different uses according to various scales that one of ordinary skill in the art will be familiar with. Generally, however, these standards define high-grade ethanol (e.g., for medical use) to be a higher purity/proof than fuel-grade ethanol (e.g., for use as a fuel or additive to another fuel).

Additionally, various materials may be referred to herein as “freed” of another material (e.g., solids-freed, solvent-freed, water-freed), indicating that the first material has been distilled, filtered, sparged, or otherwise separated to remove (or be freed of) at least a portion of the second material. For example, a base liquid containing fifty percent water and fifty percent of an organic solvent (e.g., ethanol) may be subject to a first distillation process to produce a first water-freed stream of thirty percent water and seventy percent of the organic solvent, which may be subject to a second distillation process to produce a second water freed-stream of ten percent water and ninety percent of the organic solvent. In contrast, various materials may be referred to herein as “enriched” with another material (e.g., solvent enriched), indicating that the first material has been distilled, filtered, concentrated, or otherwise supplemented to increase a concentration of the second material. Using the previous examples, the water-freed streams may also be considered to be solvent enriched streams, and the remaining base material (from which the solvent enriched streams were separated) may be considered to be water enriched streams in comparison to the respective inputs thereof.

As will be appreciated, a solvent production plant may operate in a continuous flow, or in discrete batches. Accordingly, various liquids or gasses may be transported as streams between the various elements of the plants, which may be continuously or discontinuously delivered to different elements via appropriate piping or ducting. Therefore, the present disclosure contemplates that the person of ordinary skill in the art will understand that various valves, splitters, collection vessels, and other flow-control elements may be used to selectively configure the pipes and ducts of the plant to deliver different amounts of inputs and outputs at different times. Accordingly, each of the illustrated examples of the solvent production plants described herein may represent several different plants or a single plant at different times with elements that are inactive or selectively disconnected not illustrated to better describe and highlight the elements that are active and selectively connected. Stated differently, each example, unless stated explicitly otherwise or apparent from the context, represents a configuration or arrangement of a unified embodiment of the present disclosure.

When discussing the various streams, the present disclosure contemplates that all or a portion of a described stream can be delivered between a first element and a second element. Accordingly, as used herein when discussing that an identified stream is directed between a first element and a second element, the present disclosure contemplates that at least a portion of the identified stream is being directed as described.

For example, column A that produces stream A may direct stream A exclusively to column B at a first time and then exclusively to column C at a second time. Accordingly, column A may be understood as being configured to direct stream A to column B and to column C (e.g., as time-based first/second portions) regardless of which column is currently receiving stream A. Similarly, column D that produces stream D may simultaneously direct stream D to column E and to column F, and may be understood as being configured to direct stream D to column E and to column F (e.g., as flow-based first/second portions) regardless of what percent of stream D is provided to column E or column F at a given time. Similarly, column G that produces and directs stream G only to column H, but may be throttled to provide any portion between 0-100% of a rated flow for stream G over a given time, may be understood as being configured to direct stream G to column H, regardless of what the flow rate is at any given time. Stated differently, each of these examples shall be understood to describe a first column configured to direct a stream to a second column, regardless of whether the first column is also configured to direct that stream to a third column (at the same or different times) and regardless of what portion of the stream is so directed to any column.

Accordingly, a reference to the delivery, direction, receipt, or other action performed in relation to a stream shall be understood to refer to that action in relation to some or all of the identified stream. Additionally, discussion of an action performed in relation to a stream shall be understood as occurring at a given time, and that same action may be performed continuously across several times or discontinuously (e.g., not performed or performed differently at a second time). For example, a stream may be described as being directed to element A, while a second portion of that stream is directed to element B (either contemporaneously or at a different time), and that same stream may be directed to element A from time T₁ to time T₂, but not directed to element A from time T₂ to time T₃. Similarly, a stream may be described as being directed to element A when X % of that stream is delivered to element A at time T₁ and when Y % of that stream is delivered to element A at time T₂ (where X≠Y).

Various components of the presently disclosed systems may be in fluid communication with one another, such as through piping or ducting. The terms “piping,” “pipes,” “pipework,” “ducts,” “ducting,” “ductwork,” and “plumbing” are used interchangeably herein to refer to elements used to transport various fluids and solids throughout the described plant between or through other names elements, and may include (or omit) insulation to maintain the temperature (or allow the change of temperature) of fluids carried therein. One of ordinary skill in the relevant art will be able to select the materials and gauges of the piping or ducting based on the fluid flow requirements (e.g., flow rate, temperature, pressure, chemical composition) between two elements. Additionally, based on the positions of the various elements in the described plants and desired flowrates among the various points in the plants, one of ordinary skill in the relevant art is expected to be able to route the various pipes and fluids carried therein (e.g., via valve states) to achieve fluid communication among the various components as described herein. Accordingly, the piping may be arranged to selectively configure two or more components to be in communication with one another to direct, send, receive, etc., various inputs and outputs. Two components in fluid communication with one another may be in direct fluid communication (e.g., piping or ducting directly connects the two components without intermediate components other than valves or collectors) or may have intermediate components for processing a fluid between the two components, such as filters, pumps, heaters, odor removal vessels, coolers, condensers, vaporizers, etc.

The provided system includes vapor recompression (e.g., a mechanical or thermal vapor recompression unit) to recover heat from distillation system outputs or to upgrade low-pressure driving vapor or steam to a higher medium/high-pressure for used in other systems (e.g., distillation column feeding a dehydration unit, such as a membrane). The addition of vapor recompression enables further heat recovery within a stream by increasing the condensation temperature and pressure of that stream and later using its latent heat by condensing it. Vapor recompression units compress a vapor from a lower pressure to a higher pressure. Examples include compressors driven by engines, driven by electrical energy and motors (both referred to as mechanical vapor recompression (MVR) units), or thermal vapor recompression (TVR) units that forgo electrical power and instead use motive steam to entrain and compress the lower pressure vapor to a higher pressure (e.g., a steam jet pump).

Although the examples given herein may recite one type of compressor using one type of power source, different power sources or types of compressors can be used in a system design according to the operational constraints of the system. For example, different types of low-pressure stream or vapors can be upgraded to a higher pressure using compressors (e.g., one or both of TVR or MVR compressors), and different types of high-pressure steams across the system can be used as motive fluids in addition to or alternatively to providing thermal energy. The energy savings provided by the addition of a vapor recompression unit in an organic solvent plant can be substantial when compared to the energy necessary to produce the steam that will be used in the plant.

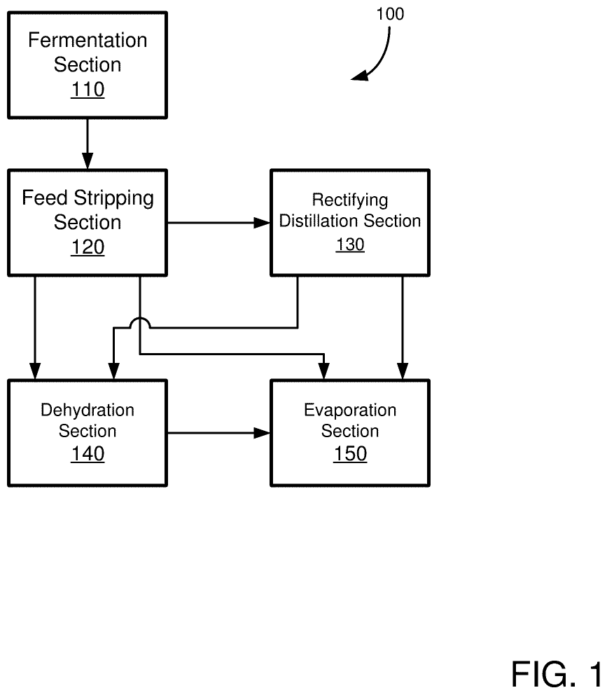

FIG. 1 illustrates an example solvent production plant 100 for an organic solvent, such as an alcohol, such as ethanol, according to aspects of the present disclosure. In at least some aspects, the provided solvent production plant 100 can be described as including five sections: a fermentation section 110, a feed stripping section 120, a rectifying distillation section 130, a dehydration section 140, and an evaporation section 150.

The fermentation section 110 includes one or more fermentation vessels (also referred to as tuns) that accept water, a vegetable matter feedstock, and a fermentation starter. The feedstock may be various sugar-bearing or cellulose-bearing vegetables or grains such as sugarcane, sugar beets, corn, switch grass, silage, or the like. Depending on the water content of the feedstock, the feedstock may be augmented with water (either fresh or from waste elsewhere in the plant). In various embodiments, the feedstock may be sliced, crushed, or cooked (e.g., to form a mash) before being fermented via a fermentation starter, which may be added after initial preparation of the feedstock and water mixture. Depending on the organic solvent to be produced by the solvent production plant 100, the fermentation starter may include various strains of bacteria or fungi (e.g., yeast) that digest the feedstock to produce a desired organic solvent (such as ethanol, methanol, isopropanol, isobutanol, acetone, etc.) and one or more metabolized waste products, such as carbon dioxide (CO₂).

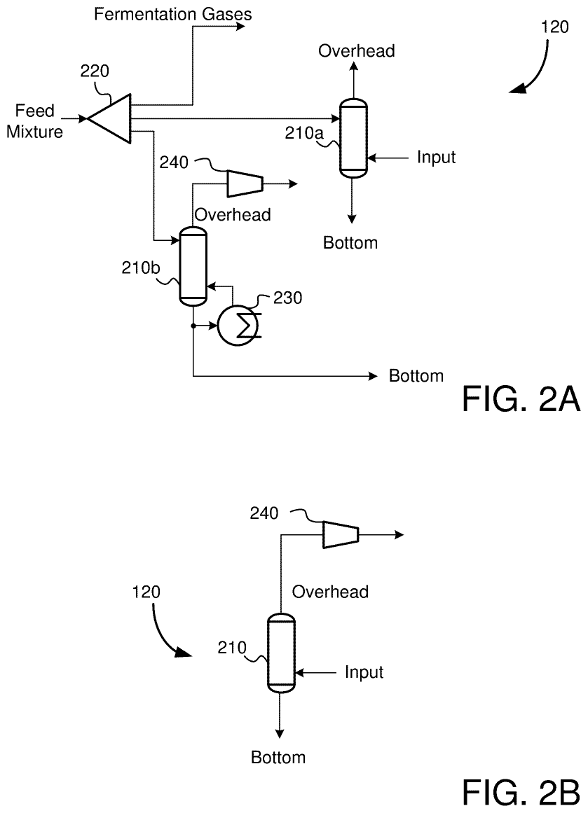

FIGS. 2A-5 discuss the components of the various sections in greater detail, and any of the various examples may be combined in the solvent production plant 100 described herein.

Various components of the presently disclosed systems may be in fluid communication with one another, such as through piping. Two components in fluid communication with one another may be in direct fluid communication (e.g., piping directly connects the two components) or may have intermediate components or processing between the two components, such as filters, pumps, heaters, odor removal vessels, etc. Various valves, valve controllers, sensors, safety features, etc. may be included in the piping to automatically or selectively control the routing of various stream through the piping to various different destinations in the solvent production plant 100, but are omitted from at least some of the Figures so as to not distract from the inventive concepts described herein.

FIGS. 2A-2B illustrate example components of a feed stripping section 120, as may be used in the solvent production plant 100 described in FIG. 1, according to embodiments of the present disclosure. FIG. 2A shows an example feed stripping section 120 that includes two distillation columns 210a-b (generally or collectively, distillation column 210), and FIG. 2B shows an example feed stripping section that includes one distillation column 210. The distillation columns 210 receive a feed stream, and optionally other input streams, to produce respective overhead stream and bottom streams to thereby distill the organic solvent from the feed stream (via heating the feed mixture into a vapor).

In various embodiments, when operating more than one distillation column 210 (as in FIG. 2A), a splitter 220 controls when and what portion of the feed stream is provided to each distillation column 210. In some embodiments, when operating more than one distillation column 210, each distillation column 210 may operate at a different pressure than the other, which may result in the different distillation columns 210 producing respective overhead streams and bottom streams with the same or different compositions between the different distillation columns 210 despite the distillation columns 210 receiving portions of a shared feed mixture.

In various embodiments, the feed mixture (also referred to as “beer” or “wine” in ethanol production) includes water, solids, and the organic solvent at a first concentration, and the overhead streams produced by the distillation columns 210 include a higher concentration of the organic solvent than the feed mixture, and the bottom streams produced by the distillation columns 210 include a higher concentration of water and solids than the feed mixture. In some embodiments, the overhead stream may be 120P ethanol, while the feed mixture has a proof below 40P.

In various embodiments, one or more of the distillation columns 210 may be associated with a reboiler 230 or other heat exchanger that recycles some or all of the bottom stream back into the distillation column 210 after exchanging heat against a heat source (such as steam or another stream within the plant to be cooled) in the heat exchanger.

In various embodiments, the overhead stream from one or more of distillation columns 210 may be compressed by a compressor 240 before being transmitted to other systems in the solvent production plant 100 as an intermediate product stream (e.g., 120P ethanol), or the distilled overhead stream can be condensed and collected in a storage tank before later distribution or use as an end-product. The present disclosure contemplates the compressor 240 may be included or omitted in various designs, and that designs using multiple distillation columns 210 may deploy a compressor in association with none, some, or all of the distillation columns 210.

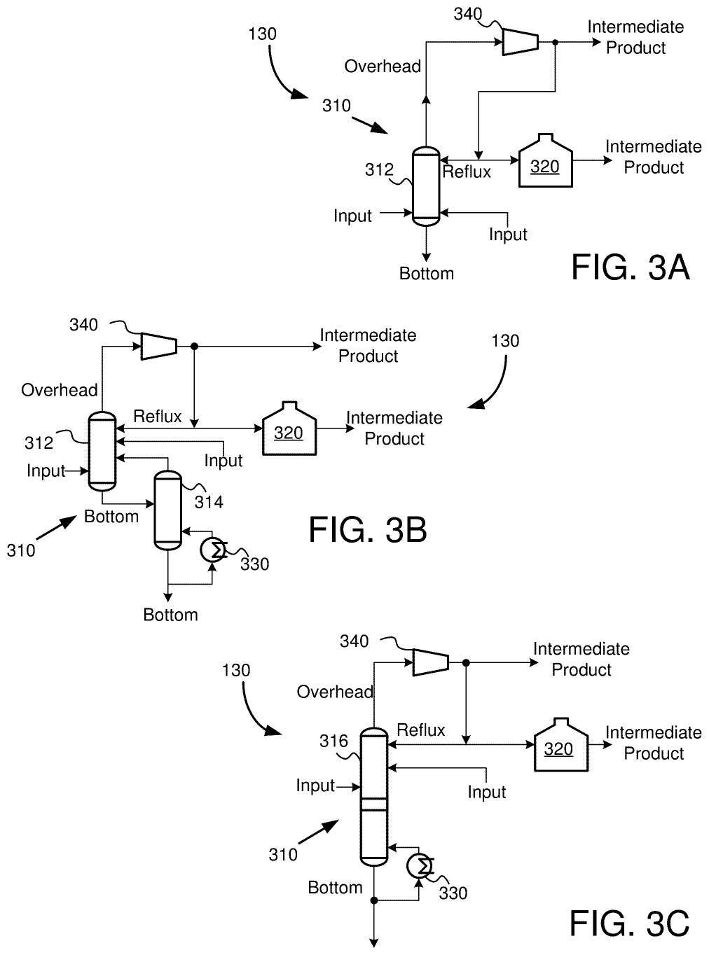

FIGS. 3A-3C illustrate example components of a rectifying distillation section 130, as may be used in the solvent production plant 100 described in FIG. 1, according to embodiments of the present disclosure. The rectifying distillation section 130 includes a rectification system 310 having at least one of: a rectifier column 312 (as in FIG. 3A), a rectifier column 312 with a side stripper column 314 (as in FIG. 3B), or an integrated column 316 (as in FIG. 3C) that includes functionalities of a rectifier and a stripper within one column (with the stripper functionality included in a lower portion of the integrated column 316).

The rectification system 310 receives input from one or more distillation columns 210, and produces an overhead stream that further increases the concentration of the organic solvent. This rectified overhead stream may be compressed by a compressor 340 before being transmitted to other systems in the solvent production plant 100 as an intermediate product stream or the rectified overhead stream can be condensed and collected in a storage tank 320 before later distribution or use as an end-product. Additionally, the rectified overhead stream may be returned to the rectification system 310 as a reflux stream, and outputs from the dehydration section 140 and evaporation section 150 may be used as supplemental inputs to the rectification system 310. In some embodiments, the overhead stream from the rectification system 310 (e.g., the rectifier column 312 or integrated column 316) may be 190P ethanol.

In embodiments including a side stripper column 314 or a stripping section in an integrated column 316, a bottom stream from the rectifier column 312 (or rectifying section in the integrated column 316) has a higher concentration of water than the overhead stream from the feed stripping section 120, and is fed into the respective side stripper column 314 or stripping section for further processing. The present disclosure also contemplates that other streams may feed into the rectifier column 312, side stripper column 314, or various sections of the integrated column 318, (e.g., scrubber bottoms, pre-condenser bottoms) to reduce losses of a solvent (e.g., ethanol) in various gas venting systems. The side stripper column 314 (or a stripping section) produces a side stripper overhead stream, which is returned to the rectifier column 312 (or rectifying section), and a side stripper bottom stream, which may be sent to other systems in the solvent production plant 100 for further processing or use as a hot or cold stream for heat exchange with another stream of a different temperature.

In various embodiments, one or more of the side stripper column 314 or stripping section of the integrated column 316 may be associated with a reboiler 330 or other heat exchanger that recycles some or all of the bottom stream back into the side stripper column 314 or stripping section of the integrated column 316 after exchanging heat against a heat source (such as steam or another stream within the plant to be cooled) in the heat exchanger.

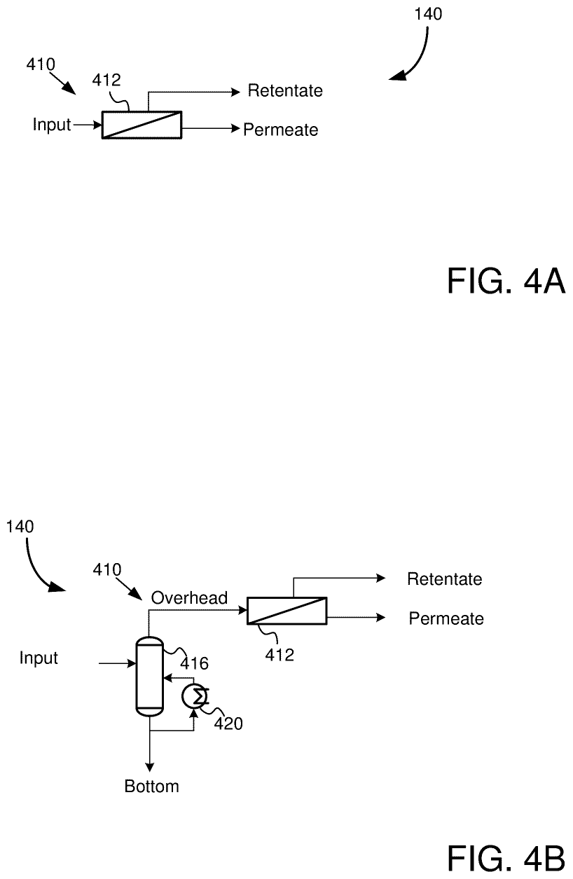

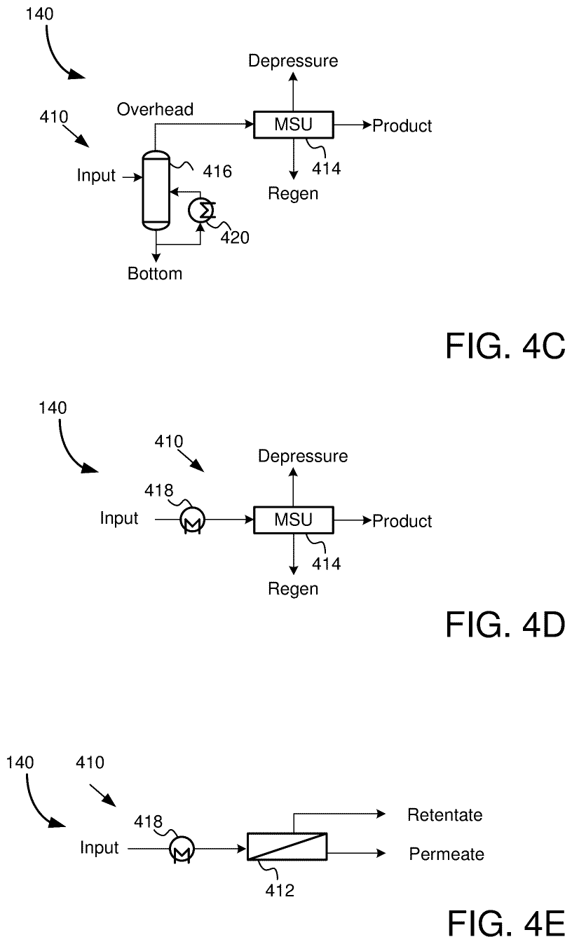

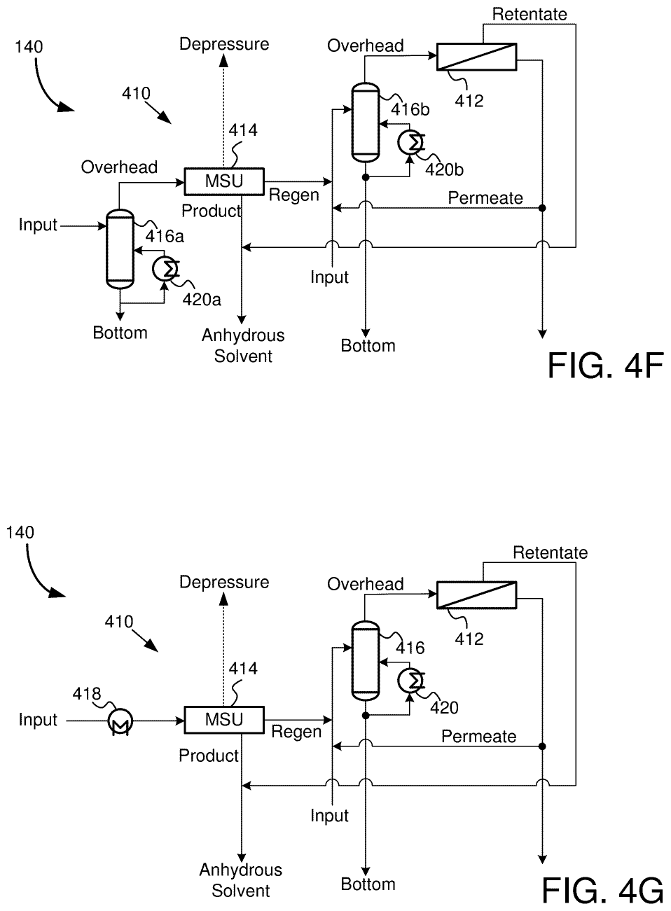

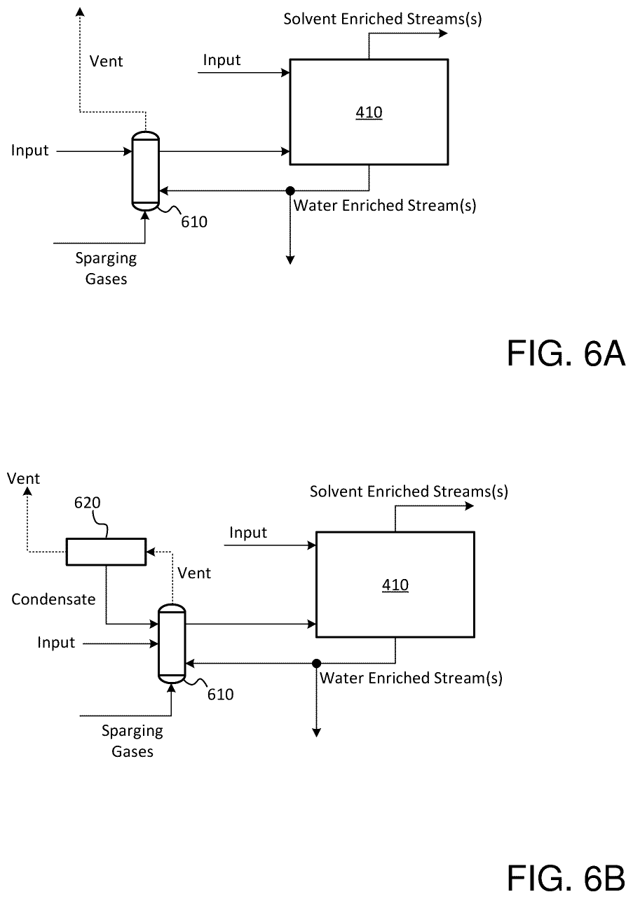

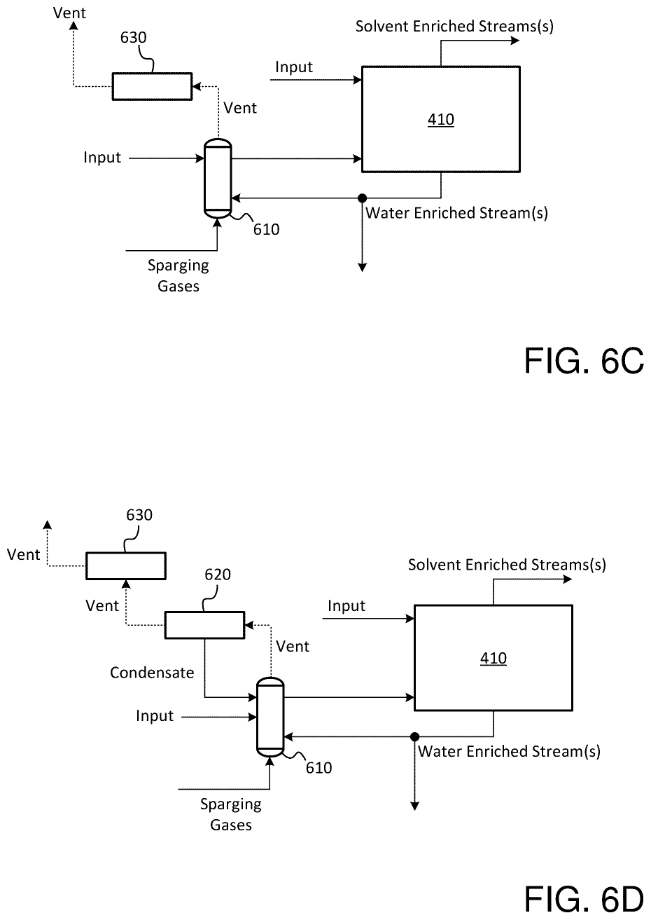

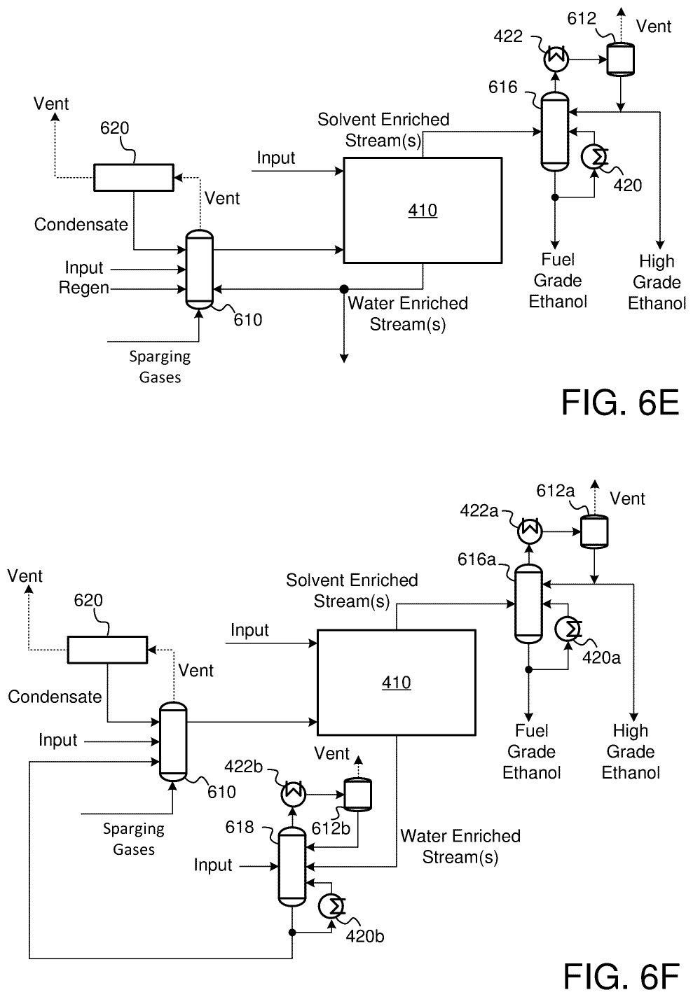

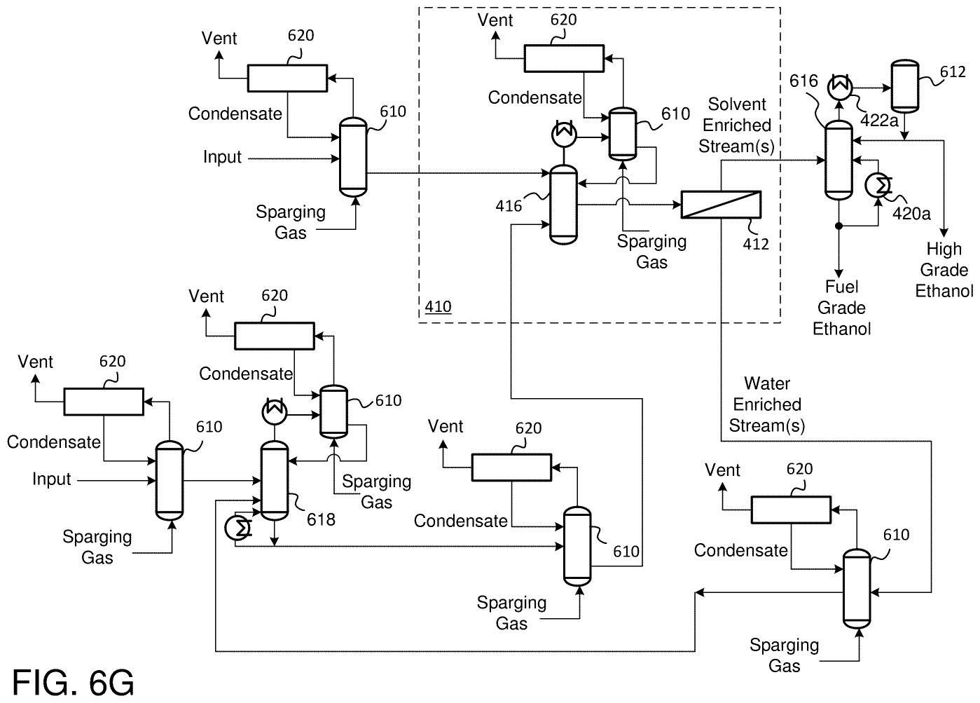

Additionally, the rectifying distillation section 130 includes various heat exchangers, splitters, flash vessels, and storage tanks, which may be arranged as illustrated in any of FIGS. 4A-4G and 6A-6G.

FIGS. 4A-4G illustrate example components of a dehydration section 140, as may be used in the solvent production plant 100 described in FIG. 1, according to embodiments of the present disclosure. The dehydration section 140 includes a separation system 410, which may include various combinations of membranes 412, molecular sieve units (MSU) 414, distillation columns 416a-b (generally or collectively, distillation column 416, and in some embodiments, a “stripper” column), and vaporizers 418 to remove water from a rectified organic solvent stream received from (at least) the rectifying distillation section 130 and produce an anhydrous organic solvent stream (e.g., a stream with a higher concentration by volume of the organic solvent due to the further removal of water from the rectified organic solvent stream). In some embodiments, the anhydrous organic solvent stream is 200P ethanol. In various embodiments, in addition or alternatively to feeding the dehydration system with a rectified organic solvent stream from the rectifying distillation section 130, the production plant 100 may provide streams of 120P, 190P, regen, depressure, rectifier bottoms, side stripper bottoms, etc., and combinations thereof as inputs.

FIG. 4A illustrates a membrane 412 deployed as a separation system 410 for the dehydration section 140. The membrane 412 continuously removes water from the input stream, to produce a permeate stream (e.g., a vaporous water-enriched stream) and a retentate stream (e.g., a vaporous anhydrous organic solvent-enriched stream). For example, the retentate stream may include 99% by volume or higher of organic solvent. In some aspects, the membrane 412 may be a polymer membrane. The polymer membrane 412 may be built on hollow fibers. A selective layer may be placed on either the outside (e.g., shell side) or the inside (e.g., lumen side) of the hollow fibers. In other examples, the membrane 412 may have other suitable forms that suitably dehydrate a feed vapor stream as part of a high-grade organic solvent production process, such as tubular membranes including zeolites membranes or spiral wound membranes.

The permeate stream may be directed to various other sections of the plant 100, or recycled back to the membrane 412 for further processing, to provide additional volume to maintain a desired flowrate through the membrane 412, to recover additional solvent from the stream, or may be directed to a heat exchanger to preheat one or more other streams in the plant 100.

FIG. 4B illustrates a membrane 412 and a distillation column 416 deployed as a separation system 410 for the dehydration section 140. The distillation column 416 may remove additional components from an input stream (e.g., via a bottom stream or the overheads stream, as a distillation column 416 may include a low boilers removal section on top, such that the feed to the membrane 412 can be drawn from a lower section in the distillation column 416) before directing an overhead stream to the membrane 412 to produce a permeate stream and a retentate stream from the overhead stream. In various embodiments, the distillation column 416 may be associated with a reboiler 420 or other heat exchanger that recycles some or all of the bottom stream back into the distillation column 416 after exchanging heat against a heat source (such as steam or another stream within the plant to be cooled) in the heat exchanger.

The permeate stream may be directed to various other sections of the plant 100, or recycled back to the distillation column 416 for further processing, to provide additional volume to maintain a desired flowrate through the membrane 412, maintain a desired fluid level in the distillation column 416, to recover additional solvent from the stream, or may be directed to a heat exchanger to preheat one or more other streams in the plant 100.

FIG. 4C illustrates an MSU 414 and a distillation column 416 deployed as a separation system 410 for the dehydration section 140. Molecular sieve dehydration involves the use of an adsorption processes using an adsorbent material in a porous solid form (known as MSUs 414) that selectively adsorb water molecules while a solvent remains a non-diffusing component due to differing molecular sizes of water and the solvent. There are many types of adsorbents, which include synthetic zeolites, microporous charcoals, active carbons, as well as natural adsorbents, including cornmeal, straw, and sawdust. Pressure swing adsorption (PSA) consists of the two main modes, which are adsorption (molecular sieve bed in online mode) and desorption (molecular sieve bed in regeneration mode).

Molecular sieve beds need to be regenerated in every cycle to be ready for the adsorption part of the cycle, which can complicate the use of MSUs 414 in continuous process systems. However, many plants may make use of several MSUs 414 in various parts of the cycle so that at least one MSU 414 is available to receive an input stream to produce a product stream (e.g., an anhydrous organic solvent-enriched stream) when in the adsorption mode, and a regen stream and/or depressure stream (e.g., a water-enriched stream) when in the desorption mode. In various embodiments, one or more solvents produced in the plant 100 (e.g., 200P ethanol) may be used to regenerate the sieve beds of the MSU 414.

The MSU 414 may include two or more beds filled with zeolite pellets, which adsorb water to produce anhydrous vapor until the zeolite pellets are saturated with water, at which point the saturated zeolite pellet bed may be regenerated. In some instances, freshly dehydrated organic solvent may be directed to contact a saturated zeolite pellet bed to remove water from the saturated zeolite pellet bed, which produces a regenerate stream. In other instances, the regeneration is done by a vacuum, thereby generating one of two regenerate streams: the regen stream and the depressure stream, depending on how the MSU is operated. The MSU regen stream may have an organic solvent concentration between 50-80 vol % and therefore is recycled to upstream distillation for reprocessing. For example, the regen stream may be directed to a distillation column 416 of the separation system.

The depressure stream (if produced) may have a concentration above 80 vol % of organic solvent, and may also be recycled to upstream distillation for further processing. For example, the depressure stream may be directed as an input to a separation system 410. In instances in which the MSU 414 includes multiple beds, a saturated bed may be regenerated while an unsaturated zeolite pellet bed is used to dehydrate a vaporized input stream (e.g., a rectifier column 312, from a rectifier/stripper column 316, from the distillation column 416). In at least some embodiments, the product stream of the MSU 414 may be condensed (e.g., via one or more evaporator systems) and directed to a tank for storage.

FIG. 4D illustrates an MSU 414 and a vaporizer 418 deployed as a separation system 410 for the dehydration section 140. In various embodiments, the vaporizer 418 may receive the input stream and heat the input stream against a heat source to vaporize the input stream for delivery to the MSU 414 in addition to or instead of a distillation column 416 producing a vaporous overhead stream for delivery to the MSU 414.

FIG. 4E illustrates membrane 412 and a vaporizer 418 deployed as a separation system 410 for the dehydration section 140. In various embodiments, the vaporizer 418 may receive the input stream and heat the input stream against a heat source to vaporize the input stream for delivery to the membrane 412 in addition to or instead of a distillation column 416 producing a vaporous overhead stream for delivery to the membrane 412.

FIG. 4F illustrates an MSU 414 with an associated first distillation column 416a and first reboiler 420a deployed in conjunction with a membrane 412 and an associated second distillation column 416b and second reboiler 420b as a separation system 410 for the dehydration section 140. In the illustrated arrangement, the first distillation column 416a receives an input stream, and produces an overhead stream that is routed to the MSU 414 to produce a product stream and a regen stream (and optionally a depressure stream), which may be provided along with other inputs to the second distillation column 416b for processing and delivery to the membrane 412 for further dehydration (or elsewhere in the plant 100 for further re-processing). In various embodiments, the product stream and the retentate stream, which are both anhydrous solvent stream (e.g., 200P ethanol) may be directed for storage or for further uses in the plant 100 (e.g., regeneration of the beds of the MSU 414, heat exchange, etc.). In various embodiments, the product stream and the retentate stream may be combined or kept separate from one another.

FIG. 4G illustrates an MSU 414 with an associated vaporizer 418 deployed in conjunction with a membrane 412 and an associated distillation column 416 and reboiler 420 as a separation system 410 for the dehydration section 140. In the illustrated arrangement, the MSU 414 receives an input stream that is preheated into a vapor by the vaporizer 418, and produces a product stream and a regen stream (and optionally a depressure stream), which may be provided along with other inputs to the second distillation column 416b for processing and delivery to the membrane 412 for further dehydration (or elsewhere in the plant 100 for further re-processing). In various embodiments, the product stream and the retentate stream, which are both anhydrous solvent stream (e.g., 200P ethanol) may be directed for storage or for further uses in the plant 100 (e.g., regeneration of the beds of the MSU 414, heat exchange, etc.). In various embodiments, the product stream and the retentate stream may be combined or kept separate from one another.

In some embodiments, the anhydrous organic solvent stream may be referred to as a retentate stream or a product stream, and may be 200P ethanol. Additionally, the dehydration section 140 includes various heat exchangers, splitters, flash vessels, and storage tanks, which may be arranged as illustrated in any of FIGS. 4A-4G and 6A-6G.

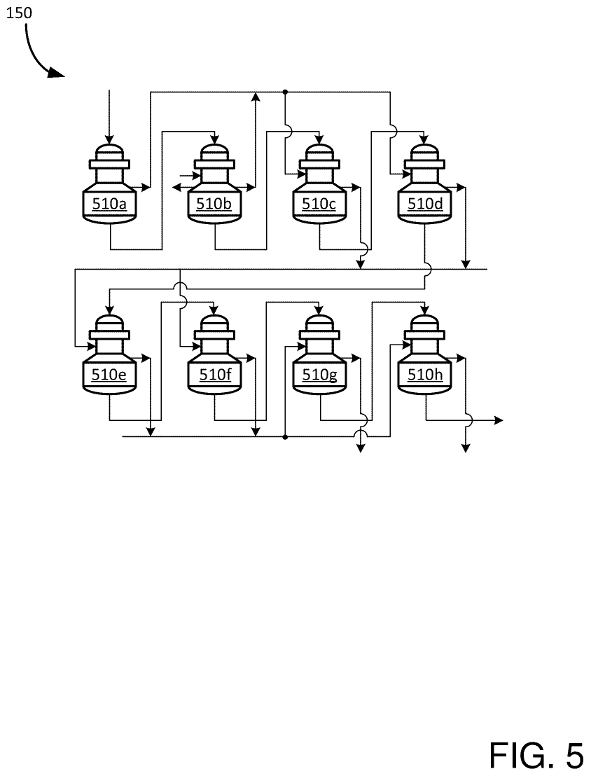

FIG. 5 illustrates example components of an evaporation section 150, as may be used in the solvent production plant 100 described in FIG. 1, according to embodiments of the present disclosure. The evaporation section 150 includes one or more evaporator systems 510a-h (generally or collectively, evaporator systems 510) that provide for the transfer of heat energy from various “hot” streams of material in the system to various “cold” streams or the environment (e.g., heat venting). In various aspects, a working fluid (e.g., water, stillage) in the evaporators extracts thermal energy from a “hot” stream, and provides that thermal energy (e.g., via steam, steam condensate, 200P vapor, 190P vapor, etc.) to another stream (e.g., the “cold” stream) in a heat exchanger.

Although illustrated in FIG. 5 with eight evaporator systems 510a-h in a cascaded arrangement, where the output of one evaporator system 510 is fed as an input to another evaporator system 510, the present disclosure contemplates that more or fewer evaporator systems 510 may be used in a solvent production plant 100 in cascaded or non-cascaded arrangements with various inputs from the other sections and outputs to the other sections. Additionally, the evaporation section 150 includes various heat exchangers, splitters, flash vessels, and storage tanks, which may be arranged as illustrated in any of FIGS. 4A-4G and 6A-6G.

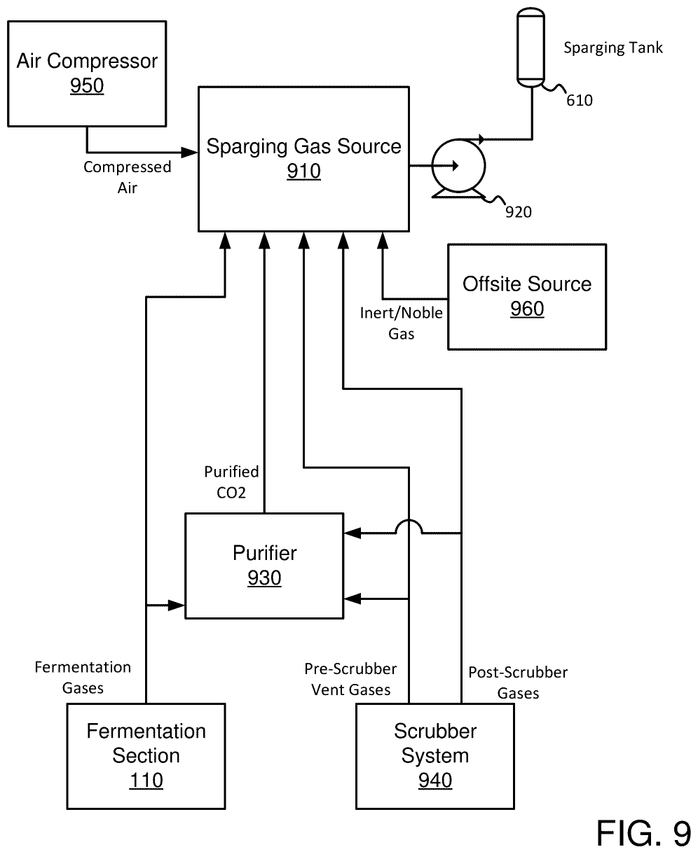

FIGS. 6A-6G illustrate example embodiments of a dehydration section 140 including a sparging tank 610, as may be used in the solvent production plant 100 described in FIG. 1, according to embodiments of the present disclosure. The sparging tank 610 provides an apparatus for the removal of congeners from a combination of a rectified organic solvent stream received from the rectifying distillation section 130 (e.g., 190P), a water enriched stream from the separation system 410 (e.g., a permeate stream exiting the membranes 412, a regen stream exiting an MSU 414, a depressure stream exiting and MSU 414, and combinations thereof), low and medium proof streams from the feed stripping section 120 and the rectifying distillation section 130 (e.g., 120P from distillation columns 210, low proof stream from the bottoms of the rectifier column 312 or the bottoms of the side stripper column 314, or the bottoms of the rectifier/stripper column 316, medium proof streams drawn from the rectifier or side stripper columns) and feeds into the distillation column 416. In various embodiments, the sparging tank 610 repurposes CO₂ produced in the fermentation section 110, and bubbles the CO₂ though the combined solution in the sparging tank 610, which increases the available surface area for mass transfer of the solution from which evaporation of congeners occurs. The sparging gases, which exit the sparging tank 610 laden with vaporous congeners and incidentally vaporized organic solvent are vented to a CO₂ scrubber (or the atmosphere) or are sent to a vent recovery system to recover solvent (e.g., ethanol) vented in the stream. Although discussed primarily in relation to using CO₂ from the fermentation section 110, as will be described in greater detail in regard to FIG. 9, other gasses and sources thereof may be used for sparging according to embodiments of the present disclosure.

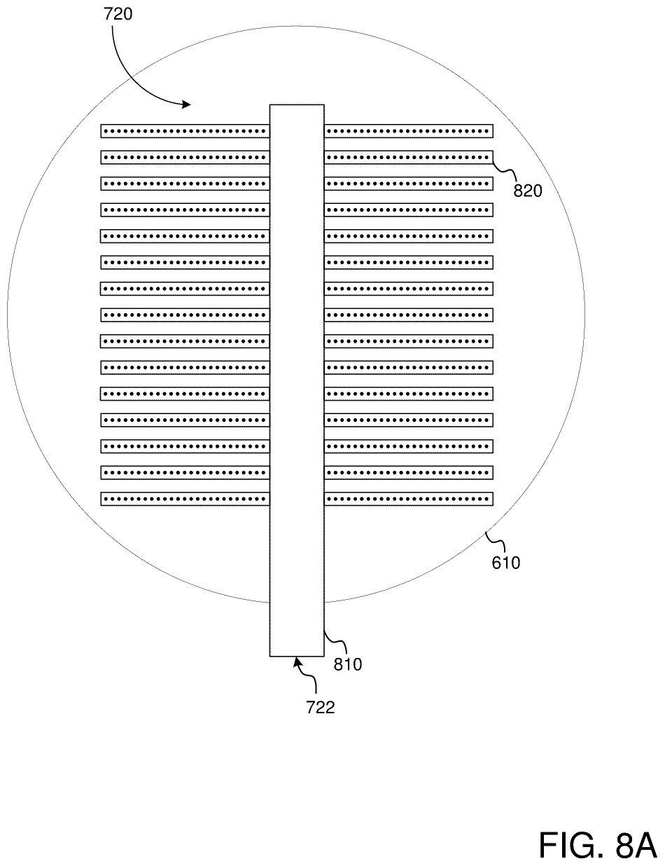

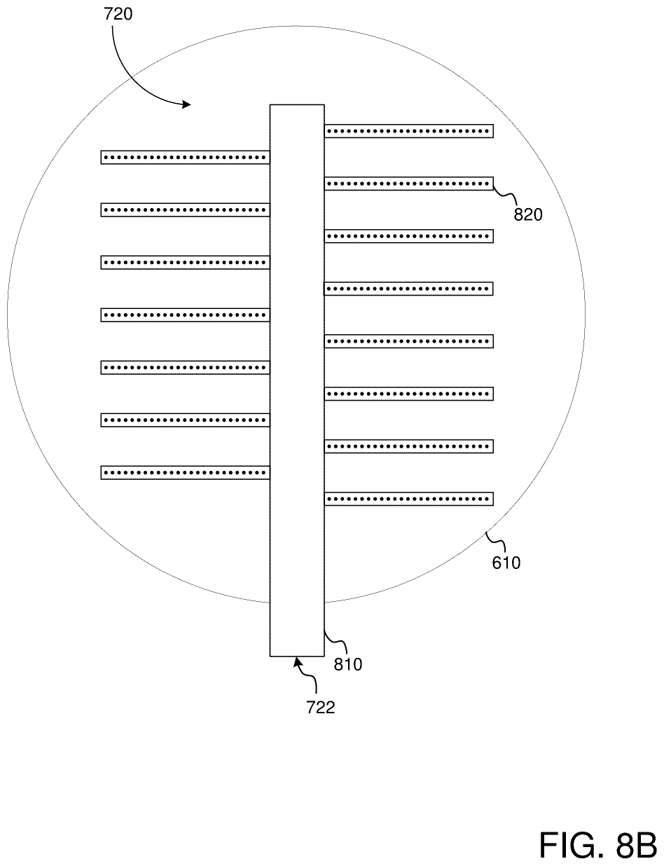

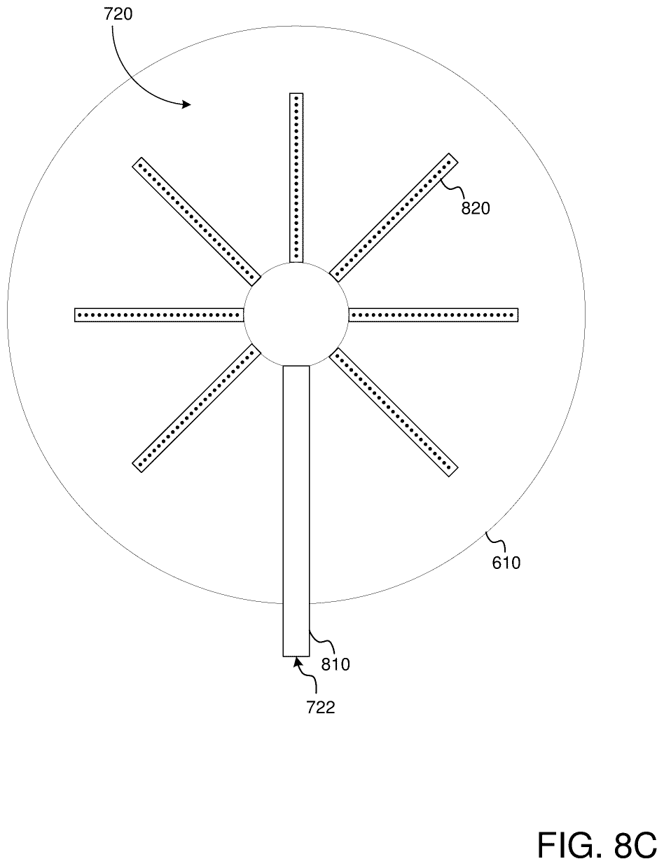

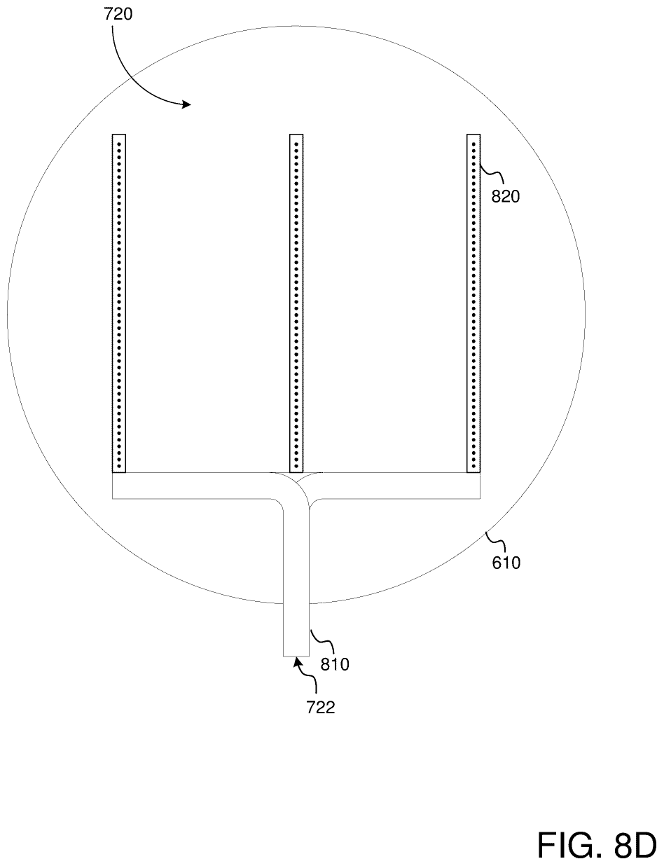

FIG. 6A illustrates an example embodiment of a dehydration section 140 including a sparging tank 610, according to embodiments of the present disclosure. Although a single sparing tank 610 is shown, the present disclosure contemplates that a solvent production plant 100 may include one or several sparging tanks 610 that are disposed in different locations of the plant 100, with various gas sources, fluid sources, and venting configurations. Generally, a sparging tank 610 receives various and variable fluid or vapor inputs, which may include regenerate streams, permeate streams, 190P streams, and other organic solvent streams having various impurity and congener contents. These various inputs are combined in the sparging tank 610, and are henceforth referred to as the “sparger feed mixture.” The sparging tank 610 includes a manifold (See FIGS. 8A-8D) that sparges inert gases, which may include CO₂ recovered from the fermentation section 110, or other gasses from other sources and combinations thereof (See FIG. 9), through the sparger feed mixture, which is held at a constant temperature below the boiling point of the organic solvent. The sparged gas bubbles increase the surface area where the sparger feed mixture contacts a gas, which in turn increases the evaporation rate of low boiling congeners from the sparger feed mixture. The increase in evaporation rate via sparging precludes a need for an increase in temperature to achieve the same increase in evaporation rate, which would increase the rate of solvent losses as the boiling point of the solvent is approached.

The inert gases, laden with vaporous congeners and incidental vaporous solvent, are vented from the sparging tank 610. In some embodiments, the sparged sparger feed mixture may be fed to a distillation column 416, where the remainder of the process is much the same as described in FIGS. 4A-4G. The distillation column 416 (e.g., a stripper column) yields overheads, which are separated into permeate and retentate streams by a membrane 412 or into regen (and optionally, depressure) and product streams by an MSU 414. In various embodiments, in addition or alternatively to using the overheads from a distillation column 416, as inputs for the membranes 412 or MSUs 414, draws may be taken from the distillation column 416 below where an overhead stream would be extracted, thereby allowing an upper section of the distillation column 416 to be used for further removal of low boiling components. The water enriched stream is outputted from the dehydration section 140 and the water enriched stream is recycled into the sparging tank 610, where the water enriched stream is combined into the sparger feed mixture. The process executed by the example embodiment of FIG. 6A is considered useful for removing volatile species when the feed temperature into the sparging tank 160 is significantly lower than the boiling point of the organic solvent.

FIG. 6B illustrates an example embodiment of a dehydration section 140 including a sparging tank 610, according to embodiments of the present disclosure. Relative to the embodiment of FIG. 6A, FIG. 6B includes a vent recovery system 620, which receives the vented gases from the sparging tank 610 and may include various condensers, separation vessels, and pumps. Contained in the vented gases is an (ideally low) concentration of vaporous solvent. The vent recovery system 620 operates at a temperature at or below the condensation point of the organic solvent, such that any incidentally vaporized organic solvent is recovered from the vent gases, which is subsequently recycled to the sparging tank 610. The process executed by the example embodiment of FIG. 6B is considered useful for removing volatile species when the feed temperature into the sparging tank 160 ranges from significantly lower than the boiling point of the organic solvent to at or above the boiling point of the organic solvent.

FIG. 6C illustrates an example embodiment of a dehydration section 140 including a sparging tank 610, according to embodiments of the present disclosure. Relative to the embodiment of FIG. 6A, FIG. 6C includes an exhauster 630, which receives the vented gases from the sparging tank 610. The exhauster 630 controls the rate at which the vent gases are vented, such that the sparging process can be performed under circumstances with controlled pressure. The pressurized process may offer advantages for the removal of certain volatile species or congeners, as evaporation rates may increase when sparging at negative pressures. The exhauster 630 vents to a CO₂ scrubber or the atmosphere. The process executed by the example embodiment of FIG. 6C is considered useful for removing volatile species when the feed temperature into the sparging tank 160 is significantly lower than the boiling point of the organic solvent and the process is to be executed under controlled pressures.

FIG. 6D illustrates an example embodiment of a dehydration section 140 including a sparging tank 610, according to embodiments of the present disclosure. FIG. 6D may be seen as a combination of the example embodiments of FIGS. 6B and 6C, as the embodiment of FIG. 6D contains both a vent recovery system 620 and an exhauster 630. The additions of the exhauster 630 and vent recovery system 620 provide the advantages discussed relative to FIGS. 6B and 6C, including organic solvent recovery and controlled pressure processes. The vented gases from the sparging tank 610 are fed to the vent recovery system 620 for solvent recovery, and the remaining gases and vaporous solvents are fed to the exhauster 630 to be vented under controlled pressure conditions. The process executed by the example embodiment of FIG. 6D is considered useful for removing volatile species when the feed temperature into the sparging tank 160 ranges from significantly lower than the boiling point of the organic solvent to at or above the boiling point of the organic solvent and the process is to be executed under controlled pressures.

FIG. 6E illustrates an example embodiment of a dehydration section 140 including a sparging tank 610, according to embodiments of the present disclosure. Relative to the embodiment of FIG. 6A, FIG. 6E includes a high boilers column 616 (as an example distillation column). While illustrated with the venting configuration of FIG. 6B, the present disclosure contemplates that that the embodiments shown in FIG. 6E can be configured to possess the venting configurations of any of the FIGS. 6A-6D. The embodiment of FIG. 6E includes a high boilers column 616, into which the retentate stream exiting the membranes 412 is fed. The high boilers column 616 yields a stream of fuel grade solvent, a portion of which may be directed into a second reboiler 420b and fed back into the high boilers column 616, and a stream of high grade solvent, which is fed into a heat exchanger 422, which feeds into a first reflux vessel 612, yielding high boiling volatile species to be vented, and a high grade ethanol stream to be outputted from the dehydration section 140. The process executed by the example embodiment of FIG. 6E is considered useful for removing volatile species when the sparger feed mixture contains levels of high boiling volatile compounds and congeners that exceed specifications.

The present disclosure also contemplates that a high boilers column 616 may receive retentate streams (e.g., from a membrane 412), product streams (e.g., from an MSU 414), and combinations thereof for the removal of high boiling components from the outputs of various separation systems.

FIG. 6F illustrates an example embodiment of a dehydration section 140 including a sparging tank 610, according to embodiments of the present disclosure. Relative to the embodiment of FIG. 6A, FIG. 6E includes a high boilers column 616 and a low boilers column 618 (as an example distillation column). While illustrated with the venting configuration of FIG. 6B, the present disclosure contemplates that the configuration shown in FIG. 6F can be reconfigured to possess the venting configurations of any of the FIGS. 6A-6D. The embodiment of FIG. 6F includes a high boilers column 616, into which the retentate stream or product stream exiting the dehydration system is fed. The high boilers column 616 yields a stream of fuel grade solvent, a portion of which may be directed into a second reboiler 420b and fed back into the high boilers column 616, and a stream of high grade solvent, which is fed into a first heat exchanger 422a, which feeds into a first reflux vessel 612a, yielding high boiling volatile species to be vented, and a high grade ethanol stream to be outputted from the dehydration section 140.

The embodiment of FIG. 6F further includes a low boilers column 618, into which the water-enriched streams exiting the dehydration section 140 are fed. In some examples, the low boilers column 618 may further receive an input of the regenerate stream. The low boilers column 618 yields an output bottoms stream having a lower concentration of low boiling volatile species and congeners than the input water-enriched stream, a portion of which may be directed to a second reboiler 420b, and recycled into the low boilers column 618. The remaining portion of the low boilers column bottoms is fed into the sparging tank 610 to be combined into the sparger feed mixture. The low boilers column 618 further yields an overheads output stream of low boiling volatile species and congeners with incidentally vaporized organic solvent, which is fed to a second heat exchanger 422b, which feeds into a second reflux vessel 612b, which yields a vent gas containing vaporous congeners and a stream of reclaimed organic solvent which is recycled into the low boilers column 618. The process executed by the example embodiment of FIG. 6F is considered useful for removing volatile species when the sparger feed mixture contains levels of both high and low boiling volatile compounds and congeners that exceed specifications.

FIG. 6G illustrates an example embodiment of a dehydration section 140 that recycle and recapture solvent while remove undesired low and high boiling components. In various embodiments, the feed sources used for the various distillation columns may include a sparging tank 610 and associated vent recovery system 620 (and optionally an exhauster 630). The low boilers column 618 is fed with different feed streams (including, optionally, a water enriched stream from the separation system 410) and one or more sparging tanks 610 are placed either in the feeds to the low boilers column 618, in the bottom stream of the low boilers column 618 feeding the separation system 410, in the reflux section on an upper portion of a respective column, or on the other streams feeding the separation system 410 (e.g., a 190P input stream).

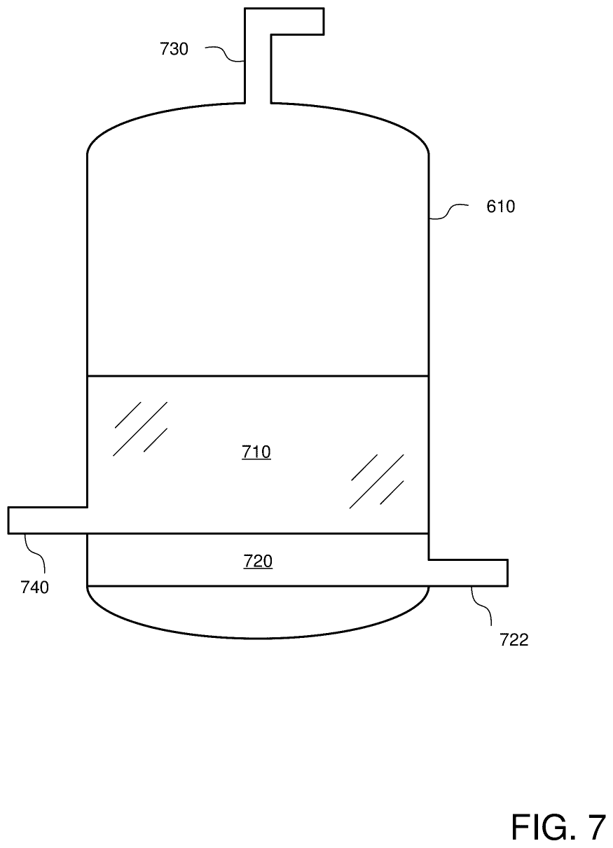

FIG. 7 illustrates a sparging tank 610, containing a distillate 710, and having a sparging manifold 720, a gas inlet 722, a distillate inlet 740, and a vent 730, according to embodiments of the present disclosure. The sparging tank 610 includes a sparging manifold 720 through which inert gases are bubbled through the distillate 710 to aid in the removal of congeners. The compositions and sources of the inert gasses are discussed in greater detail in regard to FIG. 9. In some embodiments, the sparging tank 610 may include a plurality of distillate inlets, or a single inlet from a plurality of sources. The sparging tank 610 may receive inputs of regenerate streams from the rectifying distillation section 130, permeate streams from the membranes 412 or a low boiler column 416c, distillate streams of 190P from the rectifier or distilling column, and sources of distillate containing various levels of volatile species and congeners from other portions of the plant 100.

In some embodiments, the sparging manifold 720 receives fermentation gases from the fermentation section 110 of the plant 100, where the gases are a by-product of the fermentation process. Ideally, the fermentation gases are primarily composed of CO₂. Additionally or alternatively, the gases may be received from a pre-scrubber line or a post-scrubber stream. In some embodiments, the fermentation gases may undergo a separation process to isolate the CO₂, which is then fed to the sparging manifold 720 via a blower or fan system. In some examples, the sparging manifold 720 is fed with CO₂ gas supplied from another source external to the plant 100. This disclosure further contemplates the sparging manifold 720 operating with other inert gases, such as, but not limited to, gaseous nitrogen (N₂), noble gases, and the like, to a similar effect in the sparging tank 610.

FIGS. 8A-8D illustrate various embodiments of sparging manifolds 720, according to embodiments of the present disclosure. Generally, the sparging manifolds 720 include (a) central conduit(s) 810 connected to a plurality of bubbler tubes 820. The central conduit 810 commutes the sparging gases to the bubbler tubes 820. The bubbler tubes 820 may include perforations through which gas can escape, such that when disposed in a fluid, bubbles are emitted from the perforations into the fluid. The present disclosure also contemplates that the bubbler tubes 820 may be made from sintered metal, and may have various geometries in addition to those shown in FIGS. 8A-8D.

FIG. 8A illustrates a central configuration of the sparging manifold 720, according to embodiments of the present disclosure. The central configuration includes a central conduit 810 that is aligned with a diameter of the sparging tank 610 and extends beyond the radial center of the sparging tank 610. Bubbler tubes 820 are perpendicularly connected to the central conduit 810 in a planar arrangement, and each bubbler tube 820 is aligned with another bubbler tube 820 on the opposite side of the central conduit 810.

FIG. 8B illustrates a staggered configuration of the sparging manifold 720, according to embodiments of the present disclosure. The staggered configuration consists of a central conduit 810 that is aligned with a diameter of the sparging tank 610 and extends beyond the radial center of the sparging tank 610. Bubbler tubes 820 are perpendicularly connected to the central conduit 810 in a planar arrangement, and the bubbler tubes 820 are staggered from side to side along the central conduit 810 such that each bubbler tube 820 occupies a unique longitudinal section of the central conduit 810

FIG. 8C illustrates a radial configuration of the sparging manifold 720, according to embodiments of the present disclosure. The central conduit 810 extends to the radial center of the sparging tank 610, from which a plurality of bubbler tubes 820 extend in a planar arrangement along radii of the sparging tank 610 from a central hub 830.

FIG. 8D illustrates a trident configuration of the sparging manifold 720, according to embodiments of the present disclosure. The central conduit 810 extends only partially into the sparging tank 610 and forms a T junction, such that sections 812a-b of conduit extend perpendicularly from the central conduit 810. Two bubbler tubes 820 extend perpendicularly from the ends of the sections 812a-b and a third bubbler tube 820 extends from the center of the T junction, forming a “trident” of bubbler tubes 820 extending into the sparging tank 610.

In some examples, depressure streams ejected from the MSU 414 may be commuted to the sparging tank 610 as an additional input to the sparger feed mixture. In certain cases, such as those when the depressure stream includes significant concentrations of vaporized organic solvent, it may be prudent to redirect the depressure streams to the sparging tank, and subsequently to the dehydration section 140, to recover the vaporized solvent.

FIG. 9 illustrates an inert gas sourcing scheme for use in congener removal in ethanol production, according to embedment's of the present disclosure. A sparging gas source 910 is in communication with one or more sparging tanks 610 to supply the sparging tanks 610 with one or more sparging gases. In various embodiments, a blower, condenser, or pump is disposed between the sparging gas source 910 and the sparging tank 610 to control a rate or pressure of the sparging gas supplied to the sparging tank 610. In various embodiments, the sparging gas source 910 includes one or more tanks to collect gases from various processes in the plant or the environment, which may later be selectively discharged to the sparging tanks 610.

In various embodiments, the fermentation section 110 provides fermentation gases directly for use to the sparging gas source 910, but may also direct the fermentation gases to a purifier 930 to produce purified CO₂ for use to the sparging gas source 910. In various embodiments, a scrubber system 940 in the plant 100 may provide one or both of pre-scrubber vent gases (which may contain combined streams of fermentation and other vent gases) or post-scrubber gases (e.g., scrubber column overheads) for use to the sparging gas source 910. Each of the fermentation section 110 and the scrubber system 940 may be considered to be an “on-site source” of sparging gases.

In various embodiments, the sparging gas source 910 may also use one or more offsite sources for the sparging gas, such as compressed canisters of gaseous, liquefied, or frozen sparging compounds. These inert sparging compounds may include inert and noble gases such as argon, helium, nitrogen, carbon dioxide, air, and any combination thereof.

Additionally, to process gases, the sparging gas source 910 may use ambient air, delivered by an air compressor 950, for use as a sparging gas. A purifier 930, which may include one or more filters, membranes, or adsorption beds may also be used to remove reactive or other unwanted gases from one or more input streams for the sparging gas source 910 so as to provide (for example) purified CO₂ from various solvent production processes that otherwise produce additional gases to CO₂. Depending on where the purifier 930 and the air compressor 950 are located, these sources may be considered to be “on-site” to the plant, or “offsite”.

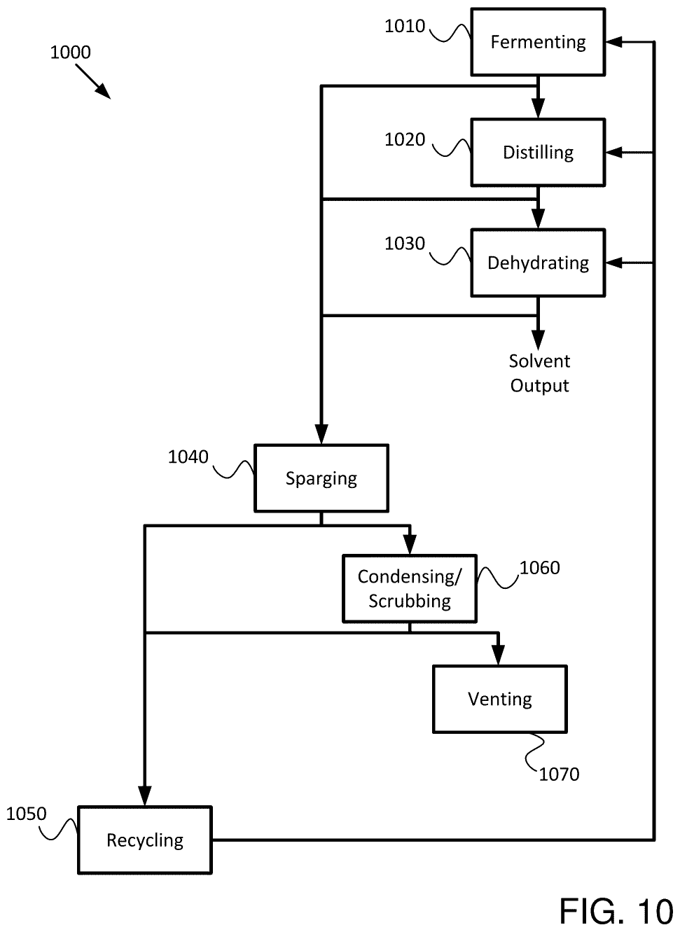

FIG. 10 illustrates a flowchart of a method 1000 for removing congeners in the production of ethanol, according to embodiments of the present disclosure.

Block 1010 of method 1000 is fermenting, wherein water, a vegetable matter feedstock, and a fermentation starter are combined to ferment into a fermentation product. The feedstock may be various sugar-bearing or cellulose-bearing vegetables or grains such as sugarcane, sugar beets, corn, switch grass, silage, or the like. Depending on the water content of the feedstock, the feedstock may be augmented with water (either fresh for from waste elsewhere in the plant). In various embodiments, the feedstock may be sliced, crushed, or cooked (e.g., to form a mash) before being fermented via a fermentation starter, which may be added after initial preparation of the feedstock and water mixture. Depending on the organic solvent to be produced by the solvent production plant 100, the fermentation starter may include various strains of bacteria or fungi (e.g., yeast) that digest the feedstock to produce a desired organic solvent (such as ethanol, methanol, isopropanol, isobutanol, acetone, etc.) and one or more metabolized waste products, such as carbon dioxide (CO₂).

According to some embodiments, the processes indicated by the fermenting block 1010 may take place in, and be conducted by the implements of, the fermentation section 110 of plant 100.

Block 1020 of method 1000 is distilling, using the fermentation product created by the processes of fermentation (e.g., block 1010). According to some embodiments, block 1020 may include multiple distillation processes. In a feed stripping distillation process, one or more distillation columns receive a feed stream of fermentation product, and optionally other input streams (which may include recycled streams from a sparging process, distillation process, rectification distillation process, dehydration process, or other processes within a solvent production plant 100), to produce respective overhead streams and bottom streams to thereby distill the organic solvent from the feed stream (via heating the feed mixture into a vapor). In various embodiments, the fermentation product includes water, solids, and the organic solvent at a first concentration, and the overhead streams produced by the distillation column(s) include a higher concentration of the organic solvent than the feed mixture, and the bottom streams produced by the distillation column(s) include a higher concentration of water than the feed mixture. In some embodiments, the overhead stream may be 120P ethanol, while the feed mixture has a proof below 60P.

According to some embodiments, in a rectifying distillation process, a rectification system receives input from one or more distillation columns, and produces an overhead stream that further increases the concentration of the organic solvent. This rectified overhead stream may be compressed by a compressor before being transmitted to other systems in a solvent production plant as an intermediate product stream or collected in a storage tank before later distribution or use as an end-product. Additionally, the rectified overhead stream may be returned to the rectification system as a reflux stream, and outputs from a dehydration section and an evaporation section may be used as supplemental inputs to the rectification system. In some embodiments, the overhead stream from the rectification system (e.g., the rectifier column or integrated column) may be 190P ethanol. In embodiments including a stripping system, a bottom stream from the rectification system is fed into the respective stripper system for further processing. The stripping system produces a side stripper overhead stream, which is returned to the rectification system, and a side stripper bottom stream, which may be sent to other systems in the solvent production plant 100 for further processing or use as a hot or cold stream for heat exchange with another stream of a different temperature.

According to some embodiments, processes indicated by the distilling block 1020 may take place in, and be conducted by the implements of, the various embodiments of the feed stripping section 120 and rectifying distillation section 130 of plant 100.

Block 1030 of method 1000 describes dehydrating, using the solvent enriched overhead streams of the distillation processes (e.g., block 1020). The dehydration process removes water from a rectified organic solvent stream received from (at least) the rectification system and produces an anhydrous organic solvent stream. In some embodiments, the anhydrous organic solvent stream is 200P ethanol. In various embodiments, other streams may also be dehydrated in addition or alternatively to the rectified organic solvent stream, such as a 120P ethanol stream, a 190P ethanol stream, recycled permeate/depressure/bottoms streams, etc.

In some embodiments, the dehydration process is executed by a dehydration system that includes a membrane that continuously removes water from the input stream, to produce a permeate stream (e.g., a vaporous water-enriched stream) and a retentate stream (e.g., a vaporous anhydrous organic solvent-enriched stream). For example, the retentate stream may include 99% by volume or higher of organic solvent. In some aspects, the membrane may be a polymer membrane. The polymer membrane may be built on hollow fibers. A selective layer may be placed on either the outside (e.g., shell side) or the inside (e.g., lumen side) of the hollow fibers. In other examples, the membrane may have other suitable forms that suitably dehydrate a feed vapor stream as part of a high-grade organic solvent production process, such as tubular membranes including zeolites membranes or spiral wound membranes. The retentate stream may be directed to various other processes, or recycled back to the membrane for further processing, to provide additional volume to maintain a desired flowrate through the membrane.

In some embodiments, the dehydration system includes a membrane and a stripper column deployed as a separation system. The stripper column may remove additional components from an input stream (e.g., via a bottom stream) before directing an overhead stream to the membrane to produce a permeate solution/stream and a retentate solution/stream from the overhead stream. In various embodiments, the stripper column may be associated with a reboiler or other heat exchanger that recycles some or all of the bottom stream back into the stripper column after exchanging heat against a heat source. The retentate stream may be directed to various other processes, or recycled back to the stripper for further processing, to provide additional volume to maintain a desired flowrate through the membrane, or maintain a desired fluid level in the stripper column.

In some embodiments, the dehydration system includes an MSU and a stripper column deployed as a separation system. Molecular sieve dehydration involves the use of an adsorption processes using an adsorbent material in a porous solid form that selectively adsorb water molecules while a solvent remains a non-diffusing component due to differing molecular sizes of water and the solvent. There are many types of adsorbents, which include synthetic zeolites, microporous charcoals, active carbons, as well as natural adsorbents, including cornmeal, straw, and sawdust. The MSU may include two or three beds filled with zeolite pellets, which adsorb water to produce anhydrous vapor until the zeolite pellets are saturated with water, at which point the saturated zeolite pellet bed may be regenerated. In some instances, freshly dehydrated organic solvent may be directed to contact a saturated zeolite pellet bed to remove water from the saturated zeolite pellet bed, which produces a regenerate stream. In other instances, the regeneration is done by a vacuum, thereby generating two regenerate streams: the regen stream and the depressure stream. The MSU regen stream may have an organic solvent concentration between 50-80 vol % and therefore is recycled to upstream distillation for reprocessing. For example, the regen stream may be directed to a stripper column of the separation system.

In some embodiments, the dehydration system includes an MSU and a vaporizer deployed as a separation system. In various embodiments, the vaporizer may receive the input stream and heat the input stream against a heat source to vaporize the input stream for delivery to the MSU in addition to or instead of a stripper column producing a vaporous overhead stream for delivery to the MSU.

In some embodiments, the dehydration system includes an MSU with an associated first stripper column and first reboiler deployed in conjunction with a membrane and an associated second stripper column and second reboiler as a separation system. In such embodiments, the first stripper column receives an input stream, and produces an overhead stream that is routed to the MSU to produce a product stream and a regen stream (and optionally a depressure stream), which may be provided along with other inputs to the second stripper column for processing and delivery to the membrane for further dehydration. In various embodiments, the product stream and the retentate stream may be combined or kept separate from one another.

In some embodiments, the dehydration system includes an MSU 414 with an associated vaporizer deployed in conjunction with a membrane and an associated stripper column and reboiler as a separation system. In such embodiments, the MSU receives an input stream that is preheated into a vapor by the vaporizer, and produces a product stream and a regen stream (and optionally a depressure stream), which may be provided along with other inputs to the second stripper column for processing and delivery to the membrane for further dehydration. In various embodiments, the product stream and the retentate stream may be combined or kept separate from one another.

According to some embodiments, the processes indicated by the dehydrating block 1030 may take place in, and be conducted by the implements of, the various embodiments of the dehydration section 140 of the plant 100.

Block 1040 of method 1000 describes sparging, using inert gases, which may include those gases from fermentation (e.g., block 1010), to purge low boiling species from the water enriched streams from distilling (e.g., block 1020) or dehydrating (e.g., block 1030) processes or a combination of such water enriched streams. The sparging process may be performed on the output of one or more of the fermentation process (e.g., per block 1010), the distilling process (e.g., per block 1020), and the dehydration process (e.g., per block 1030), and may recycle recovered solvent, water, or other captured products back to one or more of the fermentation process, the distilling process, or the dehydration process. In various embodiments, the sparging process is performed on the outputs of the same or of different processes than the solvent or other products are recycled to. In various embodiments, recycling may be omitted for some or all of the sparging systems (at least some of the time).

In some embodiments, the sparging process of block 1040 is executed by one or more sparging tanks, each including a sparging manifold. The sparging tank includes a manifold through which inert gases are bubbled through the distillate to aid in the removal of congeners and other undesirable volatile species. The sparging tank 610 may receive inputs of regenerate streams from the rectifying distillation section, permeate streams from the dehydration section, distillate streams of 190P from the rectification section, bottoms streams from various distillation columns, and sources of distillate containing various levels of volatile species and congeners.

The sparging manifold receives fermentation gases produced from the processes of the fermentation (e.g., block 1010 of method 1000), where the gases are a byproduct of the fermentation. In some embodiments, the fermentation gases are primarily composed of CO₂. In some embodiments, the fermentation gases may undergo a separation process to isolate the CO₂, which is then fed to the sparging manifold via a blower or fan system. In some examples, the sparging manifold is fed with CO₂ or another gas supplied from another external source. The present disclosure further contemplates the sparging process of block 1040 operating with other inert gases, such as, but not limited to, gaseous nitrogen (N₂), noble gases, air, and the like.

According to some embodiments, the processes indicated by the block 1040 may take place in, and be conducted by, the implements of FIGS. 6A-6F, 7 and 8A-8D.

The sparging process (e.g., block 1040) yields two output streams, a liquid stream containing water and solvent, and a gaseous stream containing vaporized low boiling congeners and other volatile species, inert sparging gas, and low concentrations of incidentally vaporized solvent. The liquid stream may be sent for recycling elsewhere in the plant, and is processed according to block 1050. The gaseous stream may be vented (per block 1070) to remove the congeners and other volatile species from the production streams. In some embodiments, the gaseous stream is vented to a vent condenser. In some embodiments the gaseous stream is vented to an exhauster. In some embodiments the gaseous stream is vented to the atmosphere.

In various embodiments, before venting (per block 1070), a condensing or scrubber process (e.g., block 1060) is performed to recapture incidentally captured solvent from the sparged stream, which is then returned for recycling and reprocessing elsewhere in the plant (e.g., per block 1050). The condensing/scrubber process of block 1060 condenses incidentally vaporized solvent out of the gaseous stream of vent gases and returns the liquid solvent to the recycling process (e.g., block 1050) for use elsewhere in the plant. In some embodiments, the condensing process of block 1060 may be omitted when the temperature of the input streams to the sparging process of block 1040 are below a certain threshold, which corresponds to lower rates of incidental solvent vaporization. According to some embodiments, the condensing process of block 1060 is included when the temperature of the input stream to the sparging process of block 1040 is above a certain threshold, which corresponds to higher rates of incidental solvent vaporization.

Block 1050 of method 1000 describes a recycling process, where the output stream from the sparging process (e.g., block 1040), is recycled into the production processes of the plant (e.g., blocks 1010-1030). The stream(s) is directed into various distillation columns or holding tanks for further processing of the recovered solvent streams, such that organic solvent is returned to the process streams, without the vented congeners and volatile species, for further isolation into a solvent-enriched stream.

As used herein, the terms low boiling species, low boiling volatile species, volatile species, low boiling congeners, low boiling odor species, and low boilers, are defined as organic species produced as a result of the fermentation process that are distinct from the desired organic solvent (e.g., ethanol), and have a boiling point that is lower than the boiling point of the desired organic solvent. Separation of low boiling species from the desired solvent in a distillate can be achieved by evaporating the low boiling species out of the distillate by raising the temperature of the mixture above the boiling points of the various low boiling species, but not to the boiling point of the solvent. The low boilers evaporate into a gaseous form from the distillate, leaving the solvent in liquid form.

As used herein, the terms high boiling species, high boiling volatile species, high boiling congener, high boiling odor species, and high boilers, are defined as organic species produced as a result of the fermentation process that are distinct from the desired organic solvent (ethanol), and have a boiling point that is higher than the boiling point of the desired organic solvent. Separation of high boiling species from the desired solvent in a distillate can be achieved by evaporating the solvent out of the distillate by raising the temperature of the distillate above the boiling point of the solvent, but not to the boiling points of the various high boiling species. The solvent evaporates into a gaseous form from the distillate, which is separated and later condensed, leaving the high boilers in liquid form.

In addition to the embodiments described above, many examples of specific combinations are within the scope of the disclosure, some of which are detailed in the clauses below:

Clause 1: A method, comprising: fermenting a feedstock in a fermentation vessel, yielding a fermentation product including an organic solvent and water, and inert fermentation gases; distilling the fermentation product in a distillation column yielding a first solvent enriched stream; dehydrating the first solvent enriched stream yielding a second solvent enriched stream having a greater concentration of the organic solvent than the first solvent enriched stream and a water enriched solution.

Clause 2: The method of any of clauses 1 or 3-11, further comprising sparging the water enriched solution with the inert fermentation gases in a sparging tank yielding a sparged solution and a vapor including volatile species present in the water enriched solution separated out from the sparged solution.

Clause 3: The method of any of clauses 1-2 or 4-11, further comprising separating the sparged solution in a stripping column and a membrane system, yielding a permeate solution and a retentate solution.

Clause 4: The method of any of clauses 1-3 or 5-11, further comprising condensing the permeate solution and recycling the permeate solution into the sparging tank.

Clause 5: The method of any of clauses 1-4 or 6-11, further comprising distilling the retentate solution.

Clause 6: The method of any of clauses 1-5 or 7-11, further comprising: condensing the permeate solution; distilling the permeate solution in a low boiler yielding a distilled permeate; and recycling the distilled permeate into the sparging tank.

Clause 7: The method of any of clauses 1-6 or 8-11, further comprising venting the vapor including the volatile species out of the sparging tank.

Clause 8: The method of any of clauses 1-7 or 9-11, wherein the vapor is vented from the sparging tank into an exhauster.

Clause 9: The method of any of clauses 1-8 or 10-11, wherein the vapor is vented into a condenser yielding a vent gas and a recovered solution, and the recovered solution is returned to the sparging tank.

Clause 10: The method of any of clauses 1-9 or 11, wherein the vent gas is vented into an exhauster.

Clause 11: The method of any of clauses 1-10, wherein the water enriched solution includes at least two of a permeate stream from a membrane used to produce a product stream of the organic solvent, a regen stream from a molecular sieve unit (MSU) used to produce the product stream of the organic solvent, and a depressure stream from the MSU.

Clause 12: A method to remove low boiling organic compounds from fuel grade or high-grade ethanol streams using an inert gas, such as carbon dioxide, existing at a plant as a gas source through a system comprising a blower, a tank with sparging network, and a vent recovery condenser.

Clause 13: The method of clause 12, wherein the method is performed under vacuum conditions using a vent exhauster system.

Clause 14: A system comprising: a fermentation vessel; a distillation column; a separation system; a tank comprising a sparging manifold; and a vent recovery condenser, wherein: the distillation column is configured to receive a fermentation product from the fermentation vessel including a solvent and water and to output a first solvent enriched solution; the separation system is configured to receive a first distillate and yield a water enriched solution; the tank is configured to receive the water enriched solution; the sparging manifold is configured to sparge the water enriched solution with an inert fermentation gas produced in the fermentation vessel as a byproduct of generating the fermentation product, yielding a saturated vapor; and the vent recovery condenser is configured to condense a solvent from the saturated vapor.

Clause 15: A system, comprising: a tank, configured to contain a fluid; a sparging apparatus; and a vent recovery system; wherein the sparging apparatus is disposed within the tank, and is configured to receive fermentation gases from a fermentation vessel, thereby sparging the fluid in the tank, forming a saturated vapor that includes a congener and a solvent, which the vent recovery system is configured to receive to thereby separate the solvent from the congener.

Certain terms are used throughout the description and claims to refer to particular features or components. As one skilled in the art will appreciate, different persons may refer to the same feature or component by different names. This document does not intend to distinguish between components or features that differ in name but not function.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112 (f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

What is claimed is:

1. A method, comprising:
fermenting a feedstock in a fermentation vessel, yielding a fermentation product including an organic solvent and water, and inert fermentation gases;
distilling the fermentation product in a distillation column yielding a first solvent enriched stream; and
dehydrating the first solvent enriched stream yielding a second solvent enriched stream having a greater concentration of the organic solvent than the first solvent enriched stream and a water enriched solution.

2. The method of claim 1, further comprising sparging a process stream solution with the inert fermentation gases in a sparging tank yielding a sparged solution and a vapor including volatile species present in the process stream solution separated out from the sparged solution, wherein the process stream is one of:
a feed stream;
a 120P ethanol stream;
a 190P ethanol stream;
an overhead stream from a distillation column; and
a bottoms stream from a distillation column.

3. The method of claim 1, further comprising sparging the water enriched solution with the inert fermentation gases in a sparging tank yielding a sparged solution and a vapor including volatile species present in the water enriched solution separated out from the sparged solution.

4. The method of claim 3, further comprising separating the sparged solution in a stripper column and a membrane system, yielding a permeate solution and a retentate solution.

5. The method of claim 4, further comprising condensing the permeate solution and recycling the permeate solution into the sparging tank.

6. The method of claim 5, further comprising distilling the retentate solution.

7. The method of claim 4, further comprising:
condensing the permeate solution;
distilling the permeate solution in a low boiler yielding a distilled permeate; and
recycling the distilled permeate into the sparging tank.

8. The method of claim 7, wherein the permeate solution is combined with at least one other feed stream between being condensed and being distilled.

9. The method of claim 3, further comprising venting the inert fermentation gases including the volatile species out of the sparging tank.

10. The method of claim 9, wherein the inert fermentation gases are vented from the sparging tank into a scrubber system, further comprising:
separating, in the scrubber system, the organic solvent from the inert fermentation gases including the volatile species; and
recovering the organic solvent from the scrubber system.

11. The method of claim 9, wherein the inert fermentation gases are vented from the sparging tank into an exhauster.

12. The method of claim 9, wherein the inert fermentation gases are vented into a condenser yielding a vent gas and a recovered solution, and the recovered solution is returned to the sparging tank.

13. The method of claim 12, wherein the vent gas is vented into an exhauster.

14. The method of claim 1, wherein the water enriched solution includes at least one of:
a permeate stream from a membrane used to produce a product stream of the organic solvent;
a regen stream from a molecular sieve unit (MSU) used to produce the product stream of the organic solvent;
a depressure stream from the MSU;
a low proof ethanol stream;
a 120P ethanol stream;
a 190P ethanol stream;
a bottoms stream; and
a scrubber water stream from a scrubber system.

15. A method to remove low boiling organic compounds from fuel grade or high-grade ethanol streams using an inert gas, such as carbon dioxide, existing at a plant or from an external source as a gas source through a system comprising a blower, a tank with sparging network, and a vent recovery system.

16. The method of claim 15, wherein the method is performed under vacuum conditions using a vent exhauster system.

17. A system, comprising:
a fermentation vessel;
a distillation column;
a separation system;
a tank comprising a sparging manifold; and
a vent recovery system,
wherein:
the distillation column is configured to receive a fermentation product from the fermentation vessel including a solvent and water and to output a first solvent enriched solution;
the separation system is configured to receive a first distillate and yield a water enriched solution;
the tank is configured to receive the water enriched solution;
the sparging manifold is configured to sparge the water enriched solution with an inert fermentation gas produced in the fermentation vessel as a byproduct of generating the fermentation product, yielding a saturated inert gas; and
the vent recovery system is configured to condense a solvent from the saturated inert gas.

18. The system of claim 17, wherein the tank receives one or more additional feeds to the water enriched solution, including:
a low proof ethanol stream from a fermentation section or a storage tank;
a 120P ethanol stream;
a 190P ethanol stream;
a bottoms stream;
a reflux stream; and
a scrubber water stream from a scrubber system.

19. A system, comprising:
a tank, configured to contain a fluid;
a sparging apparatus; and
a vent recovery system,
wherein the sparging apparatus is disposed within the tank, and is configured to receive fermentation gases from a fermentation vessel, thereby sparging the fluid in the tank, forming a saturated inert gas that includes a congener and a solvent, which the vent recovery system is configured to receive to thereby separate the solvent from the congener.
